(19) United States
(12) Patent Application Publication
(10) Pub. No.: US 2023/0007884 A1
(43) Pub. Date: Jan. 12, 2023

(54) APPARATUS AND METHOD FOR ESTIMATING BIO-INFORMATION

(71) Applicant: SAMSUNG ELECTRONICS CO., LTD., Suwon-si (KR)

(72) Inventors: Jae Min Kang, Seoul (KR); Seung Woo Noh, Seongnam-si (KR); Sang Yun Park, Hwaseong-si (KR); Jin Woo Choi, Suwon-si (KR)

(73) Assignee: SAMSUNG ELECTRONICS CO., LTD., Suwon-si (KR)

(21) Appl. No.: 17/468,234

(22) Filed: Sep. 7, 2021

(30) Foreign Application Priority Data
Jul. 7, 2021 (KR) 10-2021-0088973

(51) Int. Cl. A61B 5/022; A61B 5/00

(52) U.S. Cl. CPC A61B 5/02225; A61B 5/7246; A61B 5/742; A61B 5/6885

(57) ABSTRACT

An apparatus for estimating bio-information is provided. According to an example embodiment, the apparatus for estimating bio-information includes: a pulse wave sensor including channels, and configured to measure pulse wave signals from an object at the channels; a force sensor configured to measure a contact force applied by the object to the pulse wave sensor; and a processor configured to determine correlations between the pulse wave signals of the channels, and to estimate bio-information based on the measured pulse wave signals and the measured contact force based on the correlations satisfying a condition.

### CROSS-REFERENCE TO RELATED APPLICATION(S)

This application claims priority from Korean Patent Application No. 10-2021-0088973, filed on Jul. 7, 2021, in the Korean Intellectual Property Office, the entire disclosure of which is incorporated by reference herein for all purposes.

### BACKGROUND

### 1. Field

Example embodiments of the disclosure relate to an apparatus and a method for non-invasively estimating bio-information.

### 2. Description of Related Art

Generally, methods of non-invasively measuring blood pressure without include a method to measure blood pressure by measuring a cuff-based pressure and a method to estimate blood pressure by measuring pulse waves without the use of a cuff.

A Korotkoff-sound method is a cuff-based blood pressure measurement method, in which a pressure in a cuff wound around an upper arm is increased and blood pressure is measured by listening to the sound generated in the blood vessel through a stethoscope while decreasing the pressure in the cuff. Another cuff-based blood pressure measurement method is an oscillometric method using an automated machine, in which a cuff is wound around an upper arm, a pressure in the cuff is increased, a pressure in the cuff is continuously measured while the cuff pressure is gradually decreased, and blood pressure is measured based on a point where a change in a pressure signal is large.

Cuffless blood pressure measurement methods generally include a method of estimating blood pressure by calculating a Pulse Transit Time (PTT), and a Pulse Wave Analysis (PWA) method of estimating blood pressure by analyzing a pulse wave shape.

### SUMMARY

According to an aspect of an example embodiment, provided is an apparatus for estimating bio-information, the apparatus including: a pulse wave sensor including channels, the pulse wave sensor being configured to measure pulse wave signals from an object at the channels; a force sensor configured to measure a contact force applied by the object to the pulse wave sensor; and a processor configured to determine correlations between the pulse wave signals of the channels, and to estimate bio-information based on the measured pulse wave signals and the measured contact force based on the correlations satisfying a condition.

The channels of the pulse wave sensor may include at least one light source configured to emit light of at least one wavelength onto the object.

The processor may be further configured to extract direct current (DC) component values from the pulse wave signals of the channels, and determine the correlations between the DC component values.

The processor may be further configured to determine correlations between DC component values of pulse wave signals having a same wavelength of the channels.

With respect to pulse wave signals having at least two different wavelengths that are measured at a first channel of the channels, the processor may be further configured to determine correlations between DC component values of the pulse wave signals having the at least two different wavelengths of the first channel.

The processor may be further configured to obtain a statistical value of the determined correlations, and based on the statistical value of the correlations being less than or equal to a predetermined threshold value, the processor may be further configured to control to guide a user to re-measure the pulse wave signals.

The processor may be further configured to obtain a statistical value of the determined correlations, and based on the statistical value of the correlations being greater than or equal to a predetermined threshold value, the processor may be further configured to estimate the bio-information based on the measured pulse wave signals and the measured contact force.

The processor may be further configured to generate an oscillometric waveform envelope based on the measured pulse wave signals and the measured contact force, and estimate the bio-information by using the generated oscillometric waveform envelope.

The apparatus may further include an output interface configured to display, via a screen, an indicator indicating a position at which the object is to be placed to contact the pulse wave sensor.

The output interface may be further configured to display a text for guiding the object to apply a uniform force to the pulse wave sensor in a constant direction.

The output interface may be further configured to display at least one of an indicator for guiding a change in a reference force to be applied by the object to the pulse wave sensor during measurement of the pulse wave signals, or an indicator indicating a change in an actual force measured by the force sensor.

According to an aspect of an example embodiment, provided is a method of estimating bio-information, the method including: by using a pulse wave sensor including channels, measuring pulse wave signals from an object at the channels; by using a force sensor, measuring a contact force applied by the object to the pulse wave sensor; determining correlations between the pulse wave signals of the channels; and estimating bio-information based on the measured pulse wave signals and the measured contact force based on the correlations satisfying a condition.

The determining the correlations may include extracting direct current (DC) component values from the pulse wave signals of the channels, and determining the correlations between the DC component values.

The determining the correlations may include determining correlations between DC component values of pulse wave signals having a same wavelength of the channels.

The determining the correlations may include, with respect to pulse wave signals having at least two different wavelengths that are measured at a first channel of the channels, determining correlations between DC component values of the pulse wave signals having the at least two different wavelengths of the first channel.

The determining the correlations may include obtaining a statistical value of the determined correlations, and the method may further include, based on the statistical value of the correlations being less than or equal to a predetermined threshold value, guiding a user to re-measure the pulse wave signals.

The determining the correlations may include obtaining a statistical value of the determined correlations, and the estimating the bio-information may include, based on the statistical value of the correlations being greater than or equal to a predetermined threshold value, estimating the bio-information based on the measured pulse wave signals and the measured contact force.

The estimating the bio-information may include generating an oscillometric waveform envelope based on the measured pulse wave signals and the measured contact force, and estimating the bio-information by using the generated oscillometric waveform envelope.

The method may further include displaying, via a screen, an indicator indicating a position at which the object is to be placed to contact the pulse wave sensor.

According to an aspect of an example embodiment, provided is an electronic device including: a main body; a pulse wave sensor including channels and provided on the main body; a force sensor provided adjacent to the pulse wave sensor and configured to measure a contact force applied by an object to the pulse wave sensor; and a processor configured to determine correlations between pulse wave signals measured at the channels, and to estimate blood pressure based on the measured pulse wave signals and measured contact force based on the correlations satisfying a condition

### BRIEF DESCRIPTION OF THE DRAWINGS

The above and other aspects, features, and advantages of the disclosure will be more apparent from the following detailed description of example embodiments taken in conjunction with the accompanying drawings, in which:

FIG. 1 is a block diagram illustrating an apparatus for estimating bio-information according to an example embodiment;

FIGS. 2A, 2B, and 2C are diagrams illustrating an arrangement structure of a pulse wave sensor according to example embodiments;

FIG. 3 is a diagram illustrating a distribution of actual blood pressure values and estimated blood pressure values;

FIG. 4A is a diagram illustrating an infrared wavelength of each channel in the case where there is a large error between actual blood pressure values and estimated blood pressure values;

FIG. 4B is a diagram illustrating a green wavelength of each channel in the case where there is a large error between actual blood pressure values and estimated blood pressure values;

FIG. 5A is a diagram illustrating an infrared wavelength of each channel in the case where there is a small error between actual blood pressure values and estimated blood pressure values;

FIG. 5B is a diagram illustrating a green wavelength of each channel in the case where there is a small error between actual blood pressure values and estimated blood pressure values;

FIG. 6 is a block diagram illustrating an apparatus for estimating bio-information according to an example embodiment;

FIGS. 7A, 7B, and 7C are diagrams explaining examples of guiding a user on contact of an object with a pulse wave sensor according to example embodiments;

FIGS. 8A and 8B are diagrams explaining an example of estimating blood pressure based on oscillometry according to an example embodiment;

FIG. 9 is a flowchart illustrating a method of estimating bio-information according to an example embodiment; and

FIGS. 10, 11, and 12 are diagrams illustrating examples of an electronic device including an apparatus for estimating bio-information according to an example embodiment.

### DETAILED DESCRIPTION

Details of example embodiments are included in the following detailed description and drawings. Advantages and features of the disclosure, and a method of achieving the same will be more clearly understood from the following example embodiments described in detail with reference to the accompanying drawings. Throughout the drawings and the detailed description, unless otherwise described, the same drawing reference numerals will be understood to refer to the same elements, features, and structures.

It will be understood that, although the terms first, second, etc. may be used herein to describe various elements, these elements should not be limited by these terms. These terms are only used to distinguish one element from another. Also, the singular forms are intended to include the plural forms as well, unless the context clearly indicates otherwise. It will be further understood that when an element is referred to as “comprising” another element, the element is intended not to exclude one or more other elements, but to further include one or more other elements, unless explicitly described to the contrary. In the following description, terms such as “unit” and “module” indicate a unit for processing at least one function or operation and they may be implemented by using hardware, software, or a combination thereof.

Hereinafter, example embodiments of an apparatus and method for estimating bio-information will be described in detail with reference to the accompanying drawings. Various embodiments of the apparatus for estimating bio-information, which will be described below, may be applied to various devices, such as a portable wearable device, a smart device, and the like. In this case, examples of various devices may include various types of wearable devices, such as a smartwatch worn on the wrist, a smart band type wearable device, a headphone type wearable device, a headband type wearable device, and the like, or a mobile device such as a smartphone, a tablet PC, etc., but are not limited thereto.

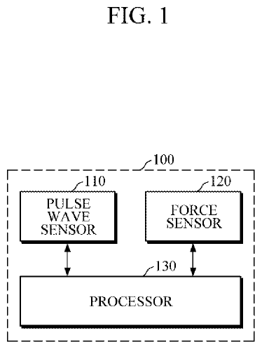

FIG. 1 is a block diagram illustrating an apparatus for estimating bio-information according to an example embodiment.

Referring to FIG. 1, an apparatus 100 for estimating bio-information includes a pulse wave sensor 110, a force sensor 120, and a processor 130.

The pulse wave sensor 110 may measure a pulse wave signal, including a photoplethysmography (PPG) signal, from an object. The pulse wave sensor 110 may include a plurality of channels. The respective channels may include one or more light sources for emitting light of one or more wavelengths onto an object, and may be disposed at different positions so as to measure pulse wave signals at different positions of the object. In this case, the one or more wavelengths may include a green wavelength, a blue wavelength, a red wavelength, an infrared wavelength, and the like. The light source may include a light emitting diode (LED), a laser diode (LD), a phosphor, etc., but is not limited thereto.

In addition, each channel of the pulse wave sensor 110 may include one or more detectors for detecting light returning after being scattered or reflected from or transmitted into a skin surface or blood vessels of the object after the light is emitted by the light source. The detector may include a photo diode, a photo transistor (PTr), an image sensor (e.g., a complementary metal-oxide semiconductor (CMOS) image sensor), etc., but is not limited thereto.

However, embodiments are not limited thereto, and one or more detectors may be disposed at a predetermined position (e.g., center of the pulse wave sensor), and the respective channels including one or more light sources may be disposed at different distances from the detectors. Alternatively, one or more light sources are disposed at a predetermined position (e.g., center of the pulse wave sensor), and the respective channels including one or more detectors may be disposed at different distances from the light sources.

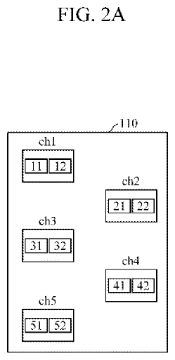

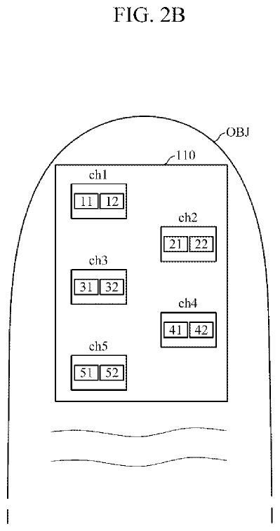

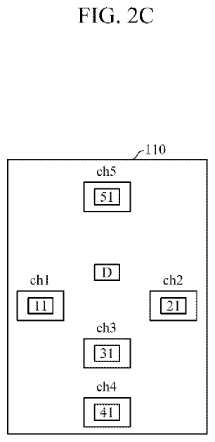

FIGS. 2A to 2C are diagrams illustrating an arrangement structure of a pulse wave sensor according to an example embodiment. Referring to FIG. 2A, the pulse wave sensor 110 includes a plurality of channels ch1, ch2, ch3, ch4, and ch5. Herein, five channels are illustrated, but the number of channels is not limited thereto. As illustrated herein, the channels ch1, ch2, ch3, ch4, and ch5 may include light sources 11, 21, 31, 41, and 51, and detectors 12, 22, 32, 42, and 52, respectively. The number of light sources and detectors included in the respective channels ch1, ch2, ch3, ch4, and ch5 is not necessarily limited to one, and may be formed as a plurality of arrays. In this case, the plurality of light sources may emit light of different wavelengths, e.g., green, blue, red, and infrared wavelengths, and the like. In addition, the respective channels ch1, ch2, ch3, ch4, and ch5 are not necessarily spaced apart from each other by an equal distance, and may be spaced apart from each other by different distances.

Referring to FIG. 2B, in the arrangement structure of the pulse wave sensor 110 as illustrated in FIG. 2A, when the object OBJ comes into contact with the pulse wave sensor 110, and a pressing force of the object OBJ gradually increases or decreases during a predetermined period of time, the respective channels ch1, ch2, ch3, ch4, and ch5 of the pulse wave sensor 110 may measure pulse wave signals from the object OBJ. The processor 130 may sequentially drive the plurality of channels ch1, ch2, ch3, ch4, and ch5, or may drive two or more channels at the same time. Among the light sources included in the channels ch1, ch2, ch3, ch4, and ch5, the processor 130 may drive only the light sources emitting light of the same wavelength, or may drive the light sources included in the same channel and emitting light of different wavelengths. Alternatively, when driving a light source of a specific channel (e.g., channel ch1), the processor 130 may drive one or more detectors of another channel (e.g., channel ch5) which are spaced apart from the light source of the specific channel.

FIG. 2C is a diagram illustrating an arrangement structure of a pulse wave sensor according to another example embodiment. Referring to FIG. 2C, one or more detectors D are disposed at the center of the pulse wave sensor 110, and the channels ch1, ch2, ch3, ch4, and ch5 may be spaced apart by an equal distance or different distances from the detectors D. The respective channels ch1, ch2, ch3, ch4, and ch5 may include one or more light sources 11, 21, 31, 41, and 51, and the one or more light sources may emit light of different wavelengths, e.g., green, blue, red, and infrared wavelengths, and the like.

The force sensor 120 may measure a force exerted on the pulse wave sensor 110 when a user places the object on the pulse wave sensor 110 and gradually increases a pressing force, or when the user applies a force greater than or equal to a threshold and then gradually decreases the force. The force sensor 120 may be disposed on an upper end or a lower end of the pulse wave sensor 110. The force sensor 120 may include a strain gauge and the like, or may be formed as a single force sensor or as an array of force sensors. In this case, the force sensor 120 may be modified to a pressure sensor in which the force sensor 120 and an area sensor are combined; an air bladder type pressure sensor, a force matrix sensor for measuring force of each pixel, or the like.

The processor 130 may be electrically connected to the pulse wave sensor 110 and/or the force sensor 120 and may control the pulse wave sensor 110 and the force sensor 120 in response to a request for estimating bio-information.

The processor 130 may determine correlations between the acquired pulse wave signals of the respective channels, may determine whether to re-measure the pulse wave signals based on the determined correlation, and may estimate bio-information based on the measured pulse wave signals and contact force. In this case, the bio-information may include heart rate, blood pressure, vascular age, arterial stiffness, aortic pressure waveform, vascular compliance, stress index, fatigue level, skin elasticity, skin age, etc., but is not limited thereto. For convenience of explanation, the following description will be made using blood pressure as an example, if necessary.

Upon receiving the pulse wave signals from the respective channels of the pulse wave sensor 110, the processor 130 may determine correlations between the received pulse wave signals. In this case, the processor 130 may determine the correlations between the pulse wave signals by using at least one of Pearson correlation, Kendall correlation, and Spearman correlation, but is not limited thereto.

The processor 130 may extract direct current (DC) component values from the pulse wave signals of the respective channels, and may determine correlations between the DC components values of the respective channels. Here, the DC component values of the respective channels may be DC component values for the same wavelength. For example, the processor 130 may determine correlations between DC component values of the pulse wave signals having a green wavelength of each channel. Generally, the DC component value may indicate a low frequency component of a signal which changes slowly over time, and may be, for example, a component in a frequency band of 0 Hz to 0.3 Hz, and a cut-off frequency (e.g., 0.3 Hz) of a low-frequency component may be adjusted according to measurement conditions. Hereinafter, a low-frequency component of the signal will be expressed as a DC component value.

Further, when the respective channels of the pulse wave sensor measure pulse wave signals of different wavelengths, the processor 130 may determine correlations between DC component values of the pulse wave signals having two or more different wavelengths of each channel. For example, the processor 130 may determine correlations between DC component values of the pulse wave signals having infrared and green wavelengths of the same channel.

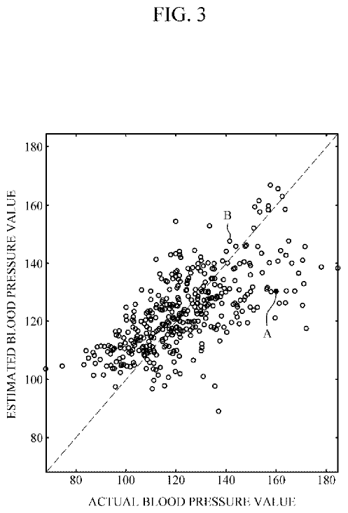

FIG. 3 is a diagram illustrating a distribution of actual blood pressure values and estimated blood pressure values. Referring to FIG. 3, if there is a large error between the actual blood pressure values and the estimated blood pressure values, the values deviate from the line y=x on a scatter plot (e.g., point A), and if there is a small error between the actual blood pressure values and the estimated blood pressure values, the values are close to the line y=x on the scatter plot (e.g., point B).

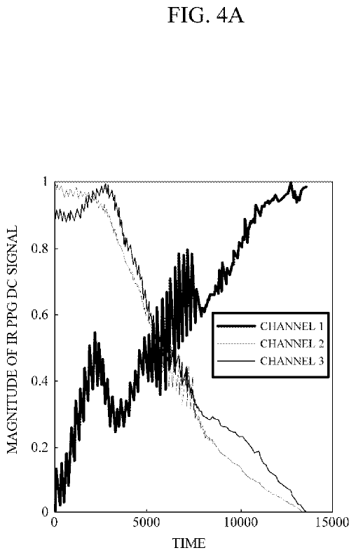

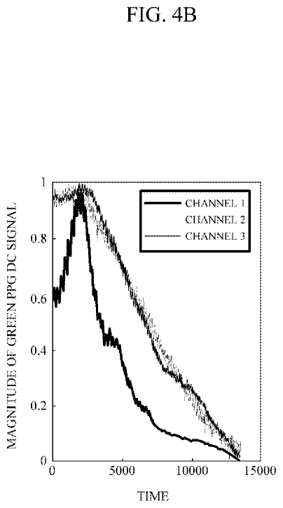

FIG. 4A is a diagram illustrating an infrared wavelength of each channel in the case where there is a large error (e.g., point A in FIG. 3) between actual blood pressure values and estimated blood pressure values; and FIG. 4B is a diagram illustrating a green wavelength of each channel in the case where there is a large error (e.g., point A in FIG. 3) between actual blood values and estimated blood pressure values. Referring to FIGS. 4A and 4B, waveforms of the pulse wave signals corresponding to channels 2 and 3 are similar, but the waveform of the pulse wave signal corresponding to channel 1 is different from the other channels (that is, channels 2 and 3), thereby resulting in a large difference in correlations between the DC component values of the pulse wave signals. This may correspond to cases where the object does not apply a uniform force to all the channels when the object comes into contact with the pulse wave sensor 110, such as a case where more pressure is applied toward channel 1 or less pressure is applied toward channel 1, and the like.

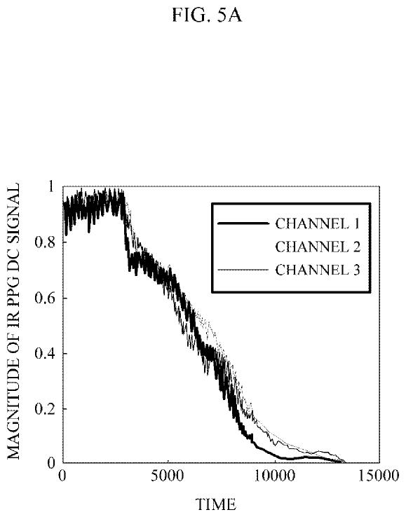

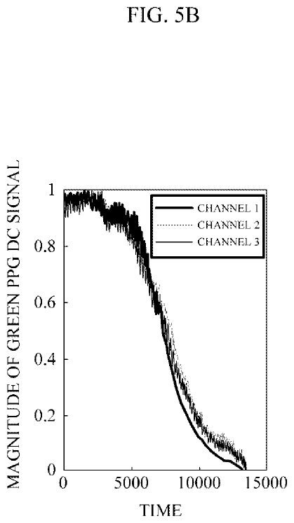

FIG. 5A is a diagram illustrating an infrared wavelength of each channel in the case where there is a small error (e.g., point B in FIG. 3) between actual blood pressure values and estimated blood pressure values; and FIG. 5B is a diagram illustrating a green wavelength of each channel in the case where there is a small error (e.g., point B in FIG. 3) between actual blood pressure values and estimated blood pressure values. Referring to FIGS. 5A and 5B, waveforms of all the pulse wave signals corresponding to channels 1, 2, and 3 are similar, thereby resulting in a small difference in correlations between the DC component values of the pulse wave signals. This may correspond to a case where the object applies a uniform force to all the channels when the object comes into contact with the pulse wave sensor 110.

As described above, by guiding measurement of pulse wave signals by using correlations between DC component values of pulse wave signals of multiple channels, and by estimating bio-information based on the pulse wave signals, accuracy of the estimation may be improved.

The processor 130 may obtain a statistical value, e.g., an average value, of the determined correlations. For example, upon obtaining DC component values of pulse wave signals of a green wavelength from the first, third, and fifth channels among the five channels, the processor 130 may determine a correlation between the DC component values of the pulse wave signals of the green wavelength for each of the first, third, and fifth channels, and may obtain an average value of the determined three correlations. Further, upon obtaining DC component values of pulse wave signals of infrared and green wavelengths from the first and second channels among the five channels, the processor 130 may determine a correlation between the DC component values of the pulse wave signals of the infrared and green wavelengths for the first channel and determine a correlation between the DC component values of the pulse wave signals of the infrared and green wavelengths for the second channel, and may obtain an average value of the determined two correlations.

The processor 130 may compare the average value of the determined correlations with a predetermined threshold value, and may determine whether to re-measure the pulse wave signals based on the comparison. For example, if the average value of the correlations is greater than or equal to the threshold value, the processor 130 may estimate bio-information based on the obtained pulse wave signals and contact force, and if the average value of the correlations is less than or equal to the threshold value, the processor 130 may guide a user to re-measure the pulse wave signals. The predetermined threshold value may refer to an average value of correlations used for distinguishing a case where the object applies a uniform force to all the channels of the pulse wave sensor 110 from a case where the object does not apply a uniform force to all the channels of the pulse wave sensor 110. For example, if an average value of the correlations is less than or equal to the threshold value, which corresponds to a case where the object does not apply a uniform force to all the channels, the processor 130 may guide a user to re-measure the pulse wave signals.

In an example embodiment of using the correlations between the pulse wave signals, the correlations between the pulse wave signals are affected by whether the object applies a uniform force to the channels, and a distance between the channels or a direction thereof does not affect the correlations between the pulse wave signals. Therefore, the channels may be freely arranged in terms of form factor.

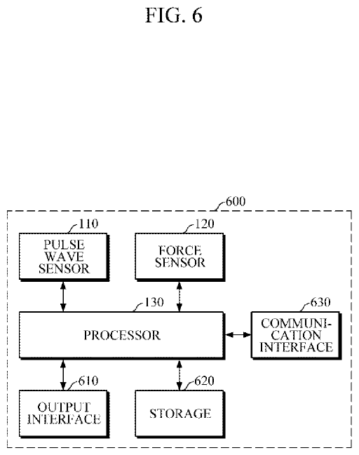

FIG. 6 is a block diagram illustrating an apparatus for estimating bio-information according to another example embodiment.

Referring to FIG. 6, an apparatus 600 for estimating bio-information according to another example embodiment may further include an output interface 610, a storage 620, and a communication interface 630, in addition to the pulse wave sensor 110, the force sensor 120, and the processor 130. The pulse wave sensor 110, the force sensor 120, and the processor 130 are described above with reference to FIG. 1, such that a description thereof will be omitted below.

The output interface 610 may output the pulse wave signal and the contact force acquired by the pulse wave sensor 110 and the force sensor 120 under the control of the processor 130, and/or various processing results of the processor 130.

For example, the output interface 610 may visually output guide information on the contact of the object, which is generated by the processor 130, through a display module, or may non-visually output the information by voice, vibrations, tactile sensation, and the like using a speaker module, a haptic module, or the like. In this case, a display area may be divided into two or more areas, in which the output interface 610 may output guide information on a contact force of the object in a first area; and may output guide information on a contact position of the object and the like in a second area. Further, the output interface 610 may output detailed information, such as the pulse wave signal, contact force, etc. used for estimating bio-information, in the form of various graphs in the first area; and along with the information, the output interface 610 may output an estimated bio-information value in the second area. In this case, if the estimated bio-information value falls outside a normal range, the output interface 610 may output warning information in various manners, such as highlighting an abnormal value in red and the like, displaying the abnormal value along with a normal range, outputting a voice warning message, adjusting a vibration intensity, and the like.

The storage 620 may store the pulse wave signal and information of the contact force acquired by the pulse wave sensor 110 and the force sensor 120 under the control of the processor 130, and/or various processing results of the processor 130. Further, the storage 620 may store a variety of reference information to be used for estimating bio-information. For example, the reference information may include user characteristic information such as a user's age, gender, health condition, etc., a bio-information estimation model, and the like, but is not limited thereto.

The storage 620 may include at least one storage medium of a flash memory type memory, a hard disk type memory, a multimedia card micro type memory, a card type memory (e.g., an SD memory, an XD memory, etc.), a Random Access Memory (RAM), a Static Random Access Memory (SRAM), a Read Only Memory (ROM), an Electrically Erasable Programmable Read Only Memory (EEPROM), a Programmable Read Only Memory (PROM), a magnetic memory, a magnetic disk, and an optical disk, and the like, but is not limited thereto.

The communication interface 630 may communicate with an external device by using wired or wireless communication techniques under the control of the processor 130, and may transmit and receive various data to and from the external device. For example, while measurement of pulse wave signals is performed, the communication interface 630 may transmit guide information on the contact of the object, which is generated by the processor 130, to the external device, so that the guide information may be displayed on a display of the external device.

Further, the communication interface 630 may transmit a bio-information estimation result, which is generated by the processor 130, to the external device and may receive, from the external device, a variety of reference information required for estimating bio-information. The external device may include a cuff-type blood pressure measuring device, and an information processing device, such as a smartphone, a tablet PC, a desktop computer, a laptop computer, and the like.

Examples of the communication techniques may include Bluetooth communication, Bluetooth Low Energy (BLE) communication, Near Field Communication (NFC), WLAN communication, Zigbee communication, Infrared Data Association (IrDA) communication, Wi-Fi Direct (WFD) communication, Ultra-Wideband (UWB) communication, Ant+ communication, WIFI communication, Radio Frequency Identification (RFID) communication, 3G communication, 4G communication, 5G communication, and the like. However, this is merely an example and is not intended to be limiting.

If both of the output interface 610 and the communication interface 630 are provided, the processor 130 may selectively control the two components 610 and 630, so that required information may be output to any one of an electronic device (e.g., smart watch), including the apparatus 600 for estimating bio-information, and an external device (e.g., smartphone). In this case, the processor 130 may determine a device to output information in response to a user's request or by using various sensors mounted in the electronic device including the apparatus 600 for estimating bio-information. For example, by using an acceleration sensor and/or a camera module, etc., mounted in the electronic device, the processor 130 may automatically detect a direction of a display mounted in the electronic device, and if the detected direction of the display is a direction (e.g., downward direction) which is beyond the reach of a user's gaze, the processor 130 may control the communication interface 630 to output information required for the external device. Alternatively, the processor 130 may control both the two components 610 and 630 so that information may be output in a mutually complementary manner.

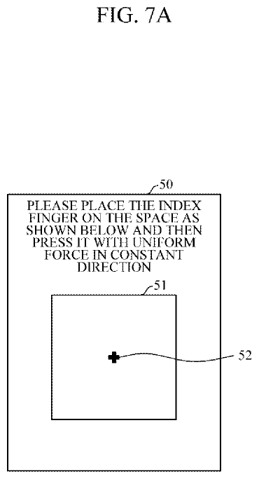

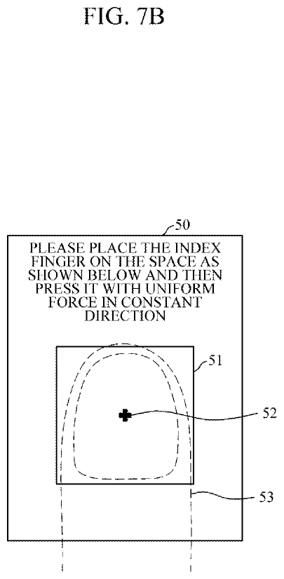

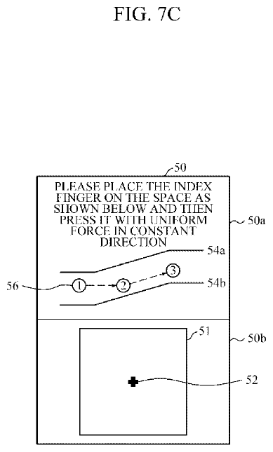

FIGS. 7A to 7C are diagrams explaining examples of guiding a user on contact of an object with a pulse wave sensor according to example embodiments.

The output interface 610 and/or the communication interface 630 may be connected to the processor 130 to display an indicator such as a graphic object having a predetermined shape on a display screen 50 of an electronic device, in which the apparatus 600 for estimating bio-information is mounted, and/or an external device, so that a user may place an object (e.g., a finger) on the pulse wave sensor. For convenience of explanation, the following description will be made based on an example in which the output interface 610 outputs the information on the display screen 50 of an electronic device in which the apparatus 600 for estimating bio-information is mounted.

Referring to FIG. 7A, upon receiving a request for estimating bio-information, the output interface 610 may display, for example, a graphic object 51, representing a space formed by the plurality of channels, so that a user may correctly place the object on the pulse wave sensor 110. A shape of the graphic object 51 may be a circle, a rectangle, a square, etc., but is not limited thereto. In addition, the output interface 610 may output a text for guiding a user to apply a contact force uniformly to the pulse wave sensor in a predetermined direction. For example, the output interface 610 may output a text, such as “please place the index finger on the space as shown below and then press it with a uniform force in a constant direction,” at an upper end of the display screen 50. Further, the output interface 610 may display a marker 52 having a predetermined shape (e.g., crisscross, circle, etc.), which is superimposed on the center of the graphic object 51, to indicate that a user is to place a feature point of the finger on the center of the square and to press vertically onto the center.

Referring to FIG. 7B, once a user's finger is placed on the pulse wave sensor 110, the processor 130 may detect a contact position and/or direction of the finger, and based on information on the detected contact position and/or direction, the output interface 610 may display a graphic object 53 having a finger shape, which is superimposed on a corresponding position of the graphic object 51.

Referring to FIG. 7C, upon receiving a request for estimating bio-information, the output interface 610 may display at least one of a graphic object for guiding a change in reference force to be applied by the object to the pulse wave sensor 110 during the measurement of pulse wave signals, and a graphic object representing a change in actual force measured by the force sensor. For example, upon receiving a request for estimating bio-information, the output interface 610 may divide the display screen 50 into two areas 50a and 50b, and may display, for example, the square graphic object 51 in a lower area 50b as described above, so that the user may correctly place the object on the space of the pulse wave sensor 110, and may display, in an upper area 50a, a graphic object representing a change in reference force, e.g., an upper limit 54a and a lower limit 54b of the reference force to be applied by the object to the pulse wave sensor 110 during the measurement time, and a graphic object 56 representing the intensity of an actual force measured by the force sensor 120. In this case, a shape of the graphic object 56 is not specifically limited, and a position of the graphic object 56 may be moved continuously, for example, in the illustrated directions 1, 2, and 3, so that a change in the actual force over time may be visually identified.

Once the processor 130 determines to re-measure the pulse wave signals, the output interface 610 may display again information of FIGS. 7A to 7C on a screen so that the user may place the object again on the pulse wave sensor 110.

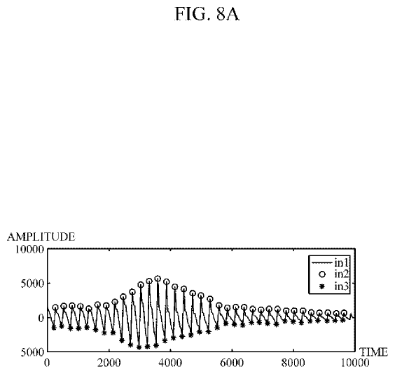

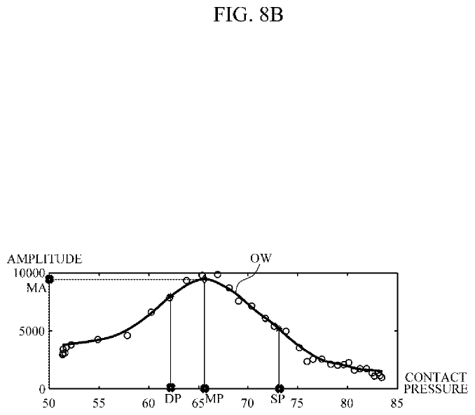

FIGS. 8A and 8B are diagrams explaining an example of estimating blood pressure based on oscillometry according to an example embodiment.

FIG. 8A illustrates a change in amplitude of a pulse wave signal when an object, being in contact with the pulse wave sensor 110, gradually increases a pressing force. FIG. 8B illustrates an oscillometric waveform envelope OW which represents a relationship between a change in contact pressure and an amplitude of the pulse wave signal. In this case, the contact pressure may be a measured force value itself, which is measured by the force sensor 120, or a value obtained by converting the force value into a pressure value by using a pre-defined conversion equation. Alternatively, in the case where a pressure sensor is mounted instead of the force sensor 120, the contact pressure may be a pressure value measured by the pressure sensor.

The processor 130 may select at least some of a plurality of channels, may generate the oscillometric waveform envelope based on pulse wave signals and contact force of the selected channels, and may estimate bio-information by using the generated oscillometric waveform envelope.

The processor 130 may extract, e.g., a peak-to-peak point of the pulse wave signal waveform by subtracting a negative (−) amplitude value in3 from a positive (+) amplitude value in2 of a waveform envelope in1 at each measurement time point of the pulse wave signal. Further, the processor 130 may obtain an oscillometic waveform envelope (OW) by plotting the peak-to-peak amplitude at each measurement time point against a contact pressure value at a corresponding time point and by performing, for example, polynomial curve fitting.

The processor 130 may estimate, for example, blood pressure by using the generated oscillometic waveform envelope OW. The processor 130 may estimate Mean Arterial Pressure (MAP) based on a contact pressure value MP at a maximum point MA of the pulse wave in the oscillogram. For example, the processor 130 may determine, as the MAP, the contact pressure value MP itself at the maximum point MA of the pulse wave, or may obtain the MAP from the contact pressure value MP by using a pre-defined MAP estimation equation. In this case, the MAP estimation equation may be expressed in the form of various linear or non-linear combination functions, such as addition, subtraction, division, multiplication, logarithmic value, regression equation, and the like, with no particular limitation.

Further, the processor 130 may estimate diastolic blood pressure and systolic blood pressure by using contact pressure values DP and SP, respectively, which are at the left and right points corresponding to amplitude values having a preset ratio, e.g., 0.5 to 0.7, to an amplitude value at the maximum point MA of the pulse wave. The processor 130 may determine the contact pressure values DP and SP as the diastolic blood pressure and systolic blood pressure, respectively, or may estimate the diastolic blood pressure and systolic blood pressure from the respective contact pressure values DP and SP by using pre-defined diastolic blood pressure and systolic blood pressure estimation equations.

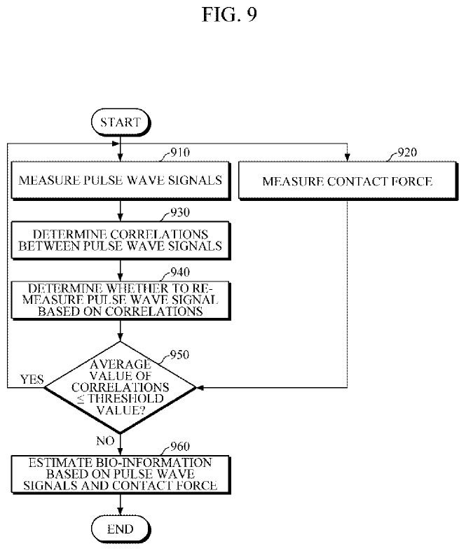

FIG. 9 is a flowchart illustrating a method of estimating bio-information according to an example embodiment.

The method of FIG. 9 may be performed by any one of the apparatuses 100 and 600 for estimating bio-information according to the embodiments of FIGS. 1 and 6, which are described above in detail, and thus will be briefly described below.

First, by using the pulse wave sensor including a plurality of channels, the apparatus for estimating bio-information may measure pulse wave signals at the respective channels from an object in 910.

By using the force sensor, the apparatus for estimating bio-information may measure a contact force applied by the object to the pulse wave sensor in 920.

Then, the apparatus for estimating bio-information may determine, in 930, correlations between the pulse wave signals of the respective channels which are acquired in 910. For example, the apparatus for estimating bio-information may extract DC component values from the pulse wave signals of the respective channels, and may determine the correlations between the DC component values of the respective channels. Further, upon determining the correlations between the DC component values of the pulse wave signals having the same wavelength of the respective channels, or upon measuring the pulse wave signals having two or more different wavelengths of the respective channels, the apparatus for estimating bio-information may determine the correlations between the DC component values of the pulse wave signals having two or more different wavelengths for each channel.

Subsequently, the apparatus for estimating bio-information may determine whether to re-measure the pulse wave signals based on the determined correlations in 940. For example, the apparatus for estimating bio-information may obtain an average value of the determined correlations to compare the obtained average value of the correlations with a predetermined threshold value, and may determine whether to re-measure the pulse wave signals based on the comparison.

If the average value of the correlations is greater than or equal to the threshold value, re-measurement is not required, and if the average value of the correlations is less than or equal to the threshold value in 950, the apparatus for estimating bio-information may proceed to the operation 910 to re-measure the pulse wave signals.

Next, if measurement of the pulse wave signals is completed such that re-measurement is not required, the apparatus for estimating bio-information may estimate bio-information based on the measured pulse wave signals and contact force in 960. For example, the apparatus for estimating bio-information may generate an oscillometric waveform envelope based on the pulse wave signals and contact force, and may estimate blood pressure by using the generated oscillometric waveform envelope. The apparatus for estimating bio-information may provide an estimated bio-information value in a visual and/or non-visual manner such as through a display, a sound output module, a haptic module, and the like. The apparatus may further output other related information in the visual and/or non-visual manner.

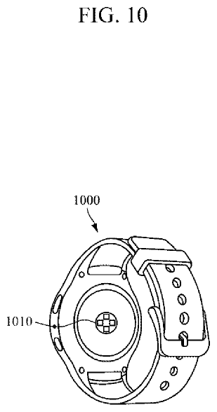

FIGS. 10 to 12 are diagrams illustrating examples of an electronic device including an apparatus for estimating bio-information according to example embodiments.

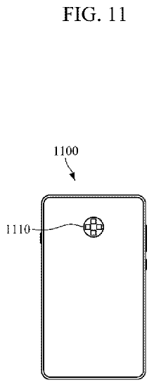

As illustrated in FIGS. 10 and 11, the electronic device may include a smart watch-type wearable device 1000 and a mobile device 1100 such as a smartphone. However, the wearable device is not limited thereto, and may include a smart band, smart glasses, a smart ring, a smart patch, a smart necklace, a tablet PC, and the like. The electronic device includes the apparatuses 100 and 600 for estimating bio-information, and all the components of the apparatuses 100 and 600 for estimating bio-information may be integrally mounted in a single device or may be distributed in two or more devices.

Referring to FIG. 10, the electronic device may be implemented as a wristwatch wearable device 1000, and may include a main body and a wrist strap. A display is provided on a front surface of the main body, and may display general application screens, including time information, received message information, etc., and/or an application screen for estimating bio-information which displays guide information on contact of an object, a blood pressure estimation result, and the like. A sensor module 1010 including the pulse wave sensor and the force sensor may be disposed on a rear surface of the main body to measure pulse wave signals and force and/or pressure for estimating bio-information from a contact portion of a user's wrist. In addition, the main body may include a processor for guiding contact of an object or estimating blood pressure by using received data, an output interface for outputting data generated by the processor on the display, and a communication interface for transmitting and receiving information by communication with other electronic devices, and the like.

Referring to FIG. 11, the electronic device may be implemented as a mobile device 1100 such as a smartphone.

The mobile device 1100 may include a housing and a display panel. The housing may form an exterior of the mobile device 1100. The housing has a first surface, on which a display panel and a cover glass may be disposed sequentially, and the display panel may be exposed to the outside through the cover glass. A sensor module 1110, a camera module and/or an infrared sensor, and the like may be disposed on a second surface of the housing. When a user transmits a request for estimating bio-information by executing an application and the like installed in the mobile device 1100, the mobile device 1100 may measure a pulse wave signal and force from an object by using the sensor module 1110. The main body may include a processor for guiding contact of an object or estimating blood pressure by using received data, an output interface for outputting data generated by the processor on a display, and a communication interface for transmitting and receiving information by communication with other electronic devices, and the like.

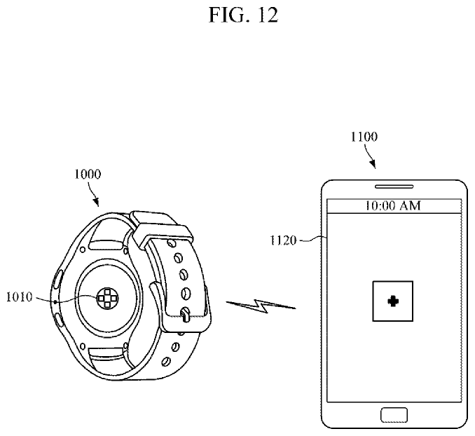

FIG. 12 illustrates an example of estimating blood pressure by interconnection between the wristwatch wearable device 1000 and the mobile device 1100. When a user estimates blood pressure by using the wearable device 1000, related information may be displayed on a display screen of the mobile device 1100. On the other hand, when a user estimates blood pressure by using the mobile device 1100, related information may be displayed on a display screen of the wearable device 1000. The wearable device 1000 may transmit guide information on contact of the object, which is generated by the processor, to the mobile device 1100 so that the information may be output on the screen of the display 1120 of the mobile device, as illustrated herein.

The disclosure may be realized as a computer-readable code written on a computer-readable recording medium. The computer-readable recording medium may be any type of recording device in which data is stored in a computer-readable manner.

Examples of the computer-readable recording medium include a ROM, a RAM, a CD-ROM, a magnetic tape, a floppy disc, an optical data storage, and a carrier wave (e.g., data transmission through the Internet). The computer-readable recording medium can be distributed over a plurality of computer systems connected to a network so that a computer-readable code is written thereto and executed therefrom in a decentralized manner. Functional programs, codes, and code segments needed for realizing the disclosure may be readily deduced by programmers of ordinary skill in the art to which the disclosure pertains.

At least one of the components, elements, modules or units described herein may be embodied as various numbers of hardware, software and/or firmware structures that execute respective functions described above, according to an example embodiment. For example, at least one of these components, elements or units may use a direct circuit structure, such as a memory, a processor, a logic circuit, a look-up table, etc. that may execute the respective functions through controls of one or more microprocessors or other control apparatuses. Also, at least one of these components, elements or units may be specifically embodied by a module, a program, or a part of code, which contains one or more executable instructions for performing specified logic functions, and executed by one or more microprocessors or other control apparatuses. Also, at least one of these components, elements or units may further include or implemented by a processor such as a central processing unit (CPU) that performs the respective functions, a microprocessor, or the like. Two or more of these components, elements or units may be combined into one single component, element or unit which performs all operations or functions of the combined two or more components, elements of units. Also, at least part of functions of at least one of these components, elements or units may be performed by another of these components, element or units. Further, although a bus is not illustrated in the block diagrams, communication between the components, elements or units may be performed through the bus. Functional aspects of the above embodiments may be implemented in algorithms that execute on one or more processors. Furthermore, the components, elements or units represented by a block or processing operations may employ any number of related art techniques for electronics configuration, signal processing and/or control, data processing and the like.

While some example embodiments have been illustrated and described above, it will be apparent to those skilled in the art that modifications and variations may be made without departing from the scope of the disclosure as defined by the appended claims and their equivalents.

What is claimed is:

1. An apparatus for estimating bio-information, the apparatus comprising:
a pulse wave sensor including channels, the pulse wave sensor being configured to measure pulse wave signals from an object at the channels;
a force sensor configured to measure a contact force applied by the object to the pulse wave sensor; and
a processor configured to:
determine correlations between the pulse wave signals of the channels, and
estimate bio-information based on the measured pulse wave signals and the measured contact force based on the correlations satisfying a condition.

2. The apparatus of claim 1, wherein the channels of the pulse wave sensor comprise at least one light source configured to emit light of at least one wavelength onto the object.

3. The apparatus of claim 1, wherein the processor is further configured to extract direct current (DC) component values from the pulse wave signals of the channels, and determine the correlations between the DC component values.

4. The apparatus of claim 3, wherein the processor is further configured to determine correlations between DC component values of pulse wave signals having a same wavelength of the channels.

5. The apparatus of claim 3, wherein the processor is further configured to, with respect to pulse wave signals having at least two different wavelengths that are measured at a first channel of the channels, determine correlations between DC component values of the pulse wave signals having the at least two different wavelengths of the first channel.

6. The apparatus of claim 3, wherein the processor is further configured to:
obtain a statistical value of the determined correlations, and
based on the statistical value of the correlations being less than or equal to a predetermined threshold value, the processor is further configured to control to guide a user to re-measure the pulse wave signals.

7. The apparatus of claim 3, wherein the processor is further configured to:
obtain a statistical value of the determined correlations, and
based on the statistical value of the correlations being greater than or equal to a predetermined threshold value, estimate the bio-information based on the measured pulse wave signals and the measured contact force.

8. The apparatus of claim 1, wherein the processor is further configured to:
generate an oscillometric waveform envelope based on the measured pulse wave signals and the measured contact force, and
estimate the bio-information by using the generated oscillometric waveform envelope.

9. The apparatus of claim 1, further comprising an output interface configured to display, via a screen, an indicator indicating a position at which the object is to be placed to contact the pulse wave sensor.

10. The apparatus of claim 9, wherein the output interface is further configured to display a text for guiding the object to apply a uniform force to the pulse wave sensor in a constant direction.

11. The apparatus of claim 9, wherein the output interface is further configured to display at least one of an indicator for guiding a change in a reference force to be applied by the object to the pulse wave sensor during measurement of the pulse wave signals, or an indicator indicating a change in an actual force measured by the force sensor.

12. A method of estimating bio-information, the method comprising:
by using a pulse wave sensor including channels, measuring pulse wave signals from an object at the channels;
measuring, by using a force sensor, a contact force applied by the object to the pulse wave sensor;
determining correlations between the pulse wave signals of the channels; and
estimating bio-information based on the measured pulse wave signals and the measured contact force based on the correlations satisfying a condition.

13. The method of claim 12, wherein the determining the correlations comprises extracting direct current (DC) component values from the pulse wave signals of the channels, and determining the correlations between the DC component values.

14. The method of claim 13, wherein the determining the correlations comprises determining correlations between DC component values of pulse wave signals having a same wavelength of the channels.

15. The method of claim 13, wherein the determining the correlations comprises, with respect to pulse wave signals having at least two different wavelengths that are measured at a first channel of the channels, determining correlations between DC component values of the pulse wave signals having the at least two different wavelengths of the first channel.

16. The method of claim 13, wherein the determining the correlations comprises obtaining a statistical value of the determined correlations,
the method further comprising, based on the statistical value of the correlations being less than or equal to a predetermined threshold value, guiding a user to re-measure the pulse wave signals.

17. The method of claim 13, wherein the determining the correlations comprises obtaining a statistical value of the determined correlations, and
wherein the estimating the bio-information comprises, based on the statistical value of the correlations being greater than or equal to a predetermined threshold value, estimating the bio-information based on the measured pulse wave signals and the measured contact force.

18. The method of claim 12, wherein the estimating the bio-information comprises generating an oscillometric waveform envelope based on the measured pulse wave signals and the measured contact force, and estimating the bio-information by using the generated oscillometric waveform envelope.

19. The method of claim 12, further comprising displaying, via a screen, an indicator indicating a position at which the object is to be placed to contact the pulse wave sensor.

20. An electronic device comprising:
a main body;
a pulse wave sensor including channels and provided on the main body;
a force sensor provided adjacent to the pulse wave sensor and configured to measure a contact force applied by an object to the pulse wave sensor; and
a processor configured to:
determine correlations between pulse wave signals measured at the channels, and
estimate blood pressure based on the measured pulse wave signals and measured contact force based on the correlations satisfying a condition.
